(19) United States
(12) Patent Application Publication
(10) Pub. No.: US 2015/0073334 A1
(43) Pub. Date: Mar. 12, 2015

(54) THERAPEUTIC DEVICE FOR ADMINISTRATION OF AEROSOL

(71) Applicants: Alexander HETZEL, Villingendorf (DE); Marc A. Reymond, Herne (DE)

(72) Inventors: Alexander HETZEL, Villingendorf (DE); Marc A. Reymond, Herne (DE)

(21) Appl. No.: 14/481,683

(22) Filed: Sep. 9, 2014

(30) Foreign Application Priority Data
Sep. 10, 2013 (DE) 10 2013 109 896.7
Sep. 10, 2013 (DE) 20 2013 104 123.8

(51) Int. Cl. A61M 5/158; A61M 5/168; A61B 17/12

(52) U.S. Cl. CPC A61M 5/158; A61B 17/12136; A61M 5/16831; A61M 2005/006
USPC 604/26

(57) ABSTRACT

A device suitable for insertion into and for use within a tubular body cavity of a patient includes a first expandable fluid chamber for sealing a first section of the body cavity, an aerosol generating apparatus for generation of a therapeutic aerosol within a treatment section of the body cavity positioned adjacent to the first section, and an insufflation apparatus for insufflation of a gaseous medium into the treatment section of the body cavity.

### CROSS REFERENCE TO RELATED APPLICATIONS

Applicants claim priority under 35 U.S.C. §119 of German Application No. 10 2013 109 896.7 filed Sep. 10, 2013 and German Application No. 20 2013 104 123.8 filed Sep. 10, 2013, the disclosures of which are incorporated by reference.

### BACKGROUND OF THE INVENTION

1. Field of the Invention

The invention relates to a medical technology device and more particularly to a device that enables application a method for local destruction of tumors in body cavities. Body cavities within the meaning of the invention include organs , especially hollow organs of humans and animals, particularly organs as esophagus, stomach, intestine, trachea, uterus, urethra, bladder, and fallopian tubes.

2. Description of the Related Art

From the WO 2012/163346 A1 a device for dispensing a substance into a body cavity is already known. The substance can be, for example, a cytostatic drug such as Doxorubicin, Cisplatin and another chemotherapeutic agent. According to the WO 2012/163346 A1 it is particularly provided that the under high pressure provided substance is mist-like dispersed in an aerosol at a needle nozzle. The obtained aerosol mist is applied to the entire surface of the body cavity reached by the mist where the substance can release its beneficial effect.

### SUMMARY OF THE INVENTION

An object of the invention is to propose an alternative apparatus for the application of the substance locally defined and highly effective within a body cavity. In particular, it is an object of the invention to utilize naturally occurring accesses of organs, especially of hollow organs, for the locally defined application of an aerosol within the organs and thereby to possibly obtain from resection procedures.

The object can be achieved by an apparatus according to the invention. Therefore, a device suitable for insertion into and for use within an organ, especially a body cavity of a patient is proposed, the device comprising a first expandable fluid chamber for sealing a first section of the body cavity, an aerosol generating apparatus for generation of a therapeutic aerosol within a treatment section positioned adjacent to the first section of the organ or body cavity.

It may furthermore be desirable to limit the effect—and thus also the often associated side effect—of the used substance to and only to areas or sections of the body cavity to be treated that actually require this effect. For example, it can be diagnosed for some types of cancers that the diseased cells to be observed do not or not yet affected the entire body cavity but that instead a localized tumor or several closely spaced tumors are present. The local restriction of the effect of the used substance is even more desirable, the more effective and thus the more toxic the selected substance is. Accordingly, in an further embodiment of the invention, the device includes a second expandable fluid chamber for sealing a second section of the body cavity, wherein the treatment section is provided as a central section between the first and the second section.

The fluid chamber may be, for example, an expandable balloon, a so called cuff or a sealable expandable tube made of an elastomer. After introduction of a fluid, such as ambient air, the fluid chamber will rest closely against the respective walls of the body cavity and might in some circumstances stretch them slightly. To enable an improved application of the effect of the aerosol, the device can include an insufflation apparatus for insufflation of a gaseous medium into the central section of the body cavity. The insufflation apparatus can be used specifically to expand the central section of the body cavity by supplying the gaseous medium at a certain pressure to the central section, such that any potential creases are evened out. CO₂ is preferably used as gaseous medium.

Especially for organs of the respiratory tract, such as the trachea and the lungs can O₂ or a mixed gas be used as gaseous medium. A pressure of a few hundredth to a few tenth provided by the insufflation apparatus has been proven to be advantageous. A pressure between about 10 mbar to about 15 mbar during the insufflation of hollow organs of the respiratory tract, especially of the trachea, and about 12 to about 15 mmHg and, thus, about 16 to about 20 mbar during the insufflation of other hollow organs has been shown to be especially advantageous. The introduction and pressurization of the gaseous medium takes place prior to the generation of the aerosol.

After the sealing by the fluid chamber and insufflation, the treatment section of the body cavity provided for therapeutic treatment can be charged with aerosol. In order to do so, the aerosol generating apparatus can be provided, for example, as a nozzle, particularly as a needle valve nozzle, which can be connected with a pump via a supply line.

In a preferred embodiment of the invention is the first fluid chamber also the only fluid chamber needed to be used. It is particularly desirable that in case of usage of the device within the trachea the fluid chamber is provided already on a known endotracheal tube or similar. The endotracheal tube may include a slightly bent and about 25-30 cm long plastic tube, which outer diameter has about the size of the diameter of a small finger of the patient. At the outlet side, the tube may include a connection piece for a ventilator or resuscitator. When using the aerosol, it should be ensured that no aerosol can exit into the ambient air. The opposite end of the tube may be beveled. About one to two finger's breadth above that can the fluid chamber in form of a small balloon, a so called cuff, be positioned. The balloon may be filled with air via a hose attached to the tube and via a valve. Thereby, gaps between the tube and the trachea can be closed whereby the trachea is sealed and the only way to the lungs is over the tube. The aerosol generating apparatus as well as the insufflation apparatus according to another embodiment can now be inserted through the above mentioned tube. Furthermore, the device may be equipped with means for positional fixation of the aerosol generating apparatus, which can be designed as a nozzle. This can be achieved according to a further embodiment of the invention, for example, by a further balloon, which is likewise finable with air via a hose that can be passed through the tube.

In a further embodiment of the invention, a device suitable for insertion into and for use within a tubular body cavity of a patient, such as, for example, the esophagus, is provided. The device comprises a first expandable fluid chamber for sealing a first section of the body cavity, a second expandable fluid chamber for sealing a second section of the body cavity, and an aerosol generating apparatus for generation of a therapeutic aerosol within a central section of the body cavity positioned between the first and the second section. The central section is in this case also the treatment section. In this embodiment, the device may further include means for insufflation of the body cavity, such as, for example, an insufflation apparatus. The insufflation apparatus may include a supply line in form of a hose that may be connected to a pressure pump that provides for example a gas or gas mixture selected from the group of CO₂, O₂ and Helium.

According to preferred embodiments of the present invention, the device can further include monitoring means suitable for monitoring the treatment section of the body cavity, wherein the monitoring means includes a camera. The camera may be equipped with means that enable rotation or other movement of the camera within the treatment section of the body cavity.

According to preferred embodiments of the present invention, the device can further include sensor means suitable for detecting the aerosol present in the treatment section. The sensor means may be, for example, an IR-diode array that detects the density of the aerosol and converts it into electrical signals. The sensor means may also be equipped with means that enable rotation or other movement of the camera within the treatment section of the body cavity.

According to preferred embodiments of the present invention, the device can further include an operation apparatus suitable for manipulation of the wall of the treatment section of the body cavity. The operation apparatus may include, for example, a cutting tool, such as biopsy forceps for removal of tissue, or tools for non-surgical use, such as tweezers.

According to preferred embodiments of the present invention, a supply line of the aerosol generating apparatus, in particular the pressure line for supplying the selected substance or a mixture of substances, is guided through the first fluid chamber into the treatment section of the body cavity. The fluid chamber may in one embodiment be formed tubular, wherein the tube is sealed relative to the supply line at both ends by utilizing suitable means, such as bonding or glueing. It was found to be especially advantages if the aerosol generating apparatus is positioned as close as possible and central to the first fluid chamber as well as along the longitudinal axis of the treatment section of the body cavity in order to administer the aerosol mist more evenly into the volume of the treatment section.

According to preferred embodiments of the present invention, at least one additional supply line is guided through the first fluid chamber into the treatment section of the body cavity. It is especially advantageous to possibly pass all of the utilized supply lines including the fluid lines as well as the electrical lines or mechanical connections to the already listed parts and the still to be listed parts through the first fluid chamber, and in particular to guide all supply lines within the tube formed within the fluid chamber.

According to preferred embodiments of the present invention, all supply lines may be guided within an accommodation means, such as a sheath tube, which is connected with the first fluid chamber in a sealed manner.

Furthermore, according to the advantageous embodiments of the present invention, the device includes an intubation apparatus, wherein a supply line of the intubation apparatus is guided through the first fluid chamber of the body cavity. This makes it possible, for example during a treatment of the trachea, to maintain the ventilation of the patient during the therapeutic aerosol therapy with the device according to embodiments of the invention.

According to further preferred embodiments of the present invention, the length of at least one of the fluid chambers is adjustable using adjustment means. By adjusting the length of the fluid chamber, the outer diameter of the fluid chamber can be better adapted to the inner cross section of the body cavity. The adjustment means may be, for example, a telescopic device or wire. In particular, also the distance between two fluid chambers can be changeable by the adjustment means.

According to further advantageous embodiments of the present invention, a device for drug therapy of a patient is provided, the device comprises one or two fluid chambers and the device is suitable for executing a method comprising the steps of:
inserting the device into a body cavity of a patient,
introducing a fluid to a first fluid chamber,
insufflating a treatment section with a gaseous medium,
introducing a fluid to a second fluid chamber, and
introducing an aerosol to the treatment section of the body cavity positioned between the fluid chambers.

If a device with just one fluid chamber is used, the step of introducing of the fluid to the second fluid naturally does not apply.

Further proposed is a device that includes an outlet means having at least an opening cross-section of the insufflation apparatus and a nozzle of the aerosol generating apparatus, wherein the outlet means opens into the treatment section. According to an embodiment, the outlet means including its connections may be passed through a tube into the treatment section. The tube includes at its outer circumferential surface the fluid chamber, for example in form of a cuff. The form of the outlet means is limited by its outer diameter according to the to be treated organs of the patient as well as the necessary dimensions of the aerosol generating apparatus and further supply lines, especially their number and diameter. In particular, the outlet means may have the shape of a flat cylinder, whereby the front face of the cylinder that faces the treatment section is formed either flat or convex. Advantageously, the outlet means is rounded to possibly prevent injury of the to be treated organs. In a particular embodiment, the outlet means can be attached in or to a hose that is passed through the fluid chamber by plugging-in, glueing or other fixation methods. Furthermore, the outlet means and the hose may be provided as a single piece according to an alternative embodiment.

The above-mentioned outlet means includes further at least one further passage leading into the treatment section. This at least one additional passage may be used for example for surgery tools, for sensors or for the supply or drainage of fluid, such as fluid of gaseous media. Advantageously is inner diameter of the passage adapted to the outer diameter of the items to be passed through. Furthermore, a pressure resistant sealing of the passage towards the treatment section may be provided by utilizing an sealing means, such as o-rings.

Finally, the first fluid chamber can be feedable through a trocar in accordance with a further embodiment of the invention. In this embodiment it is ensured that the aerosol doe not enter into undesirable section within the organ or the environment.

### BRIEF DESCRIPTION OF THE DRAWINGS

The present invention will now be described, by way of example, with reference to the accompanying drawings. It shows schematically:

FIG. 1 a first therapeutic device in perspective view,

FIGS. 2 to 4 each a sectional view of a body cavity with a top view of a further device,

FIGS. 5 and 6 each a sectional view of a fluid chamber,

FIG. 7 a sectional view of a body cavity with a top view of a further device,

FIGS. 8 and 9 each various groups of parts belonging to a device,

FIGS. 10 and 11 each a sectional view of a body cavity with a top view of a further device,

FIG. 12 a device positioned within the esophagus and stomach,

FIG. 13 a device positioned within the bladder, and

FIGS. 14 and 15 a device positioned within the trachea.

### DETAILED DESCRIPTION OF PREFERRED EMBODIMENTS

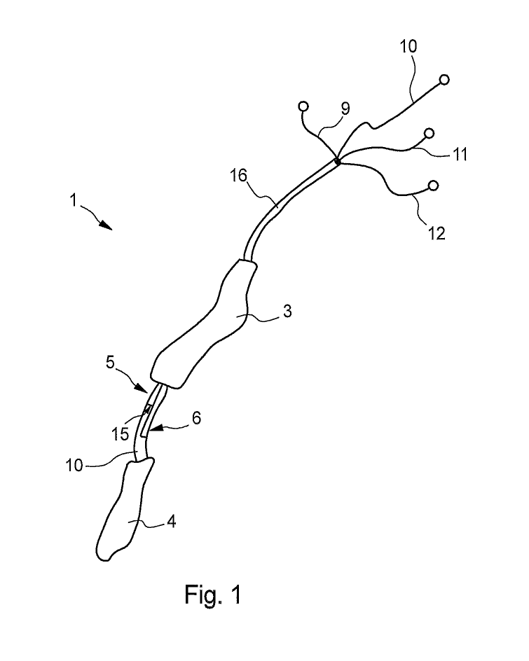

The in FIG. 1 illustrated device 1 includes a first expandable fluid chamber 3, a second expandable fluid chamber 4, an aerosol generating apparatus 5 in the form of a nozzle being connected with a supply line in form of a pressure line 11 and having an inner diameter of 20 μm for generation of a therapeutic aerosol, as well as an insufflation apparatus 6 in the form of a tube having an opening that is arranged approximately centered between the two fluid chambers 3, 4. Shown are a total of four supply lines 9-12; one supply line 9, 10 to each of the two fluid chambers, respectively, for the introduction of ambient air under relatively low pressure (about 0.8 bar); the pressure line 11 filled with a substance, preferably a chemotherapeutic substance, for delivery to the nozzle 15 under a pressure of about 20 bar; and the further air supply line 12 to the insufflation apparatus for expansion of a treatment section of a body cavity, as shown in further detail below. At least the supply lines 11 and 12 are positioned in a common sheath tube 16 and with this passed through the fluid chamber 3. The supply line 9 for the upper fluid chamber 3 may also be passed through the sheath tube 16 or may reach together with the sheath tube 16 into the fluid chamber 3. Alternatively, the fluid or air supply for the upper fluid chamber 3 can directly be connected with the fluid chamber 3 via supply line 9. The shown device and all further described devices may in accordance with embodiment of the invention be designed for one time usage. A cleaning of the devices can be eliminated in this case.

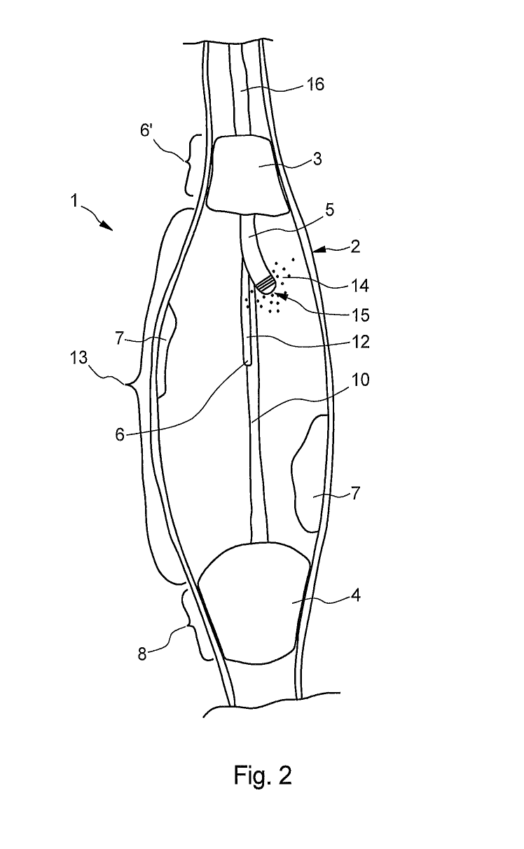

The in FIG. 2 illustrated device 1 is shown as inserted into a tubular body cavity 2 of a patient. A first fluid chamber 3 in an inflated state seals a first upper section 6′ of the body cavity 2, here the trachea. A second expandable fluid chamber 4 seals a second lower section 8 of the body cavity 2, here the esophagus. Furthermore, an aerosol generating apparatus 5 dispenses a therapeutic aerosol 14 within a treatment section 13 that extends between the first section 6′ and the second section 8 of the body cavity 2. Also shown are two tumors 7, which are devitalized by means of the aerosol 14. All supply lines except the supply line to the fluid chamber 3 are shown passing through the fluid chamber 3 within a sheath tube 16. The supply line to fluid chamber 3 ends within the fluid chamber 3.

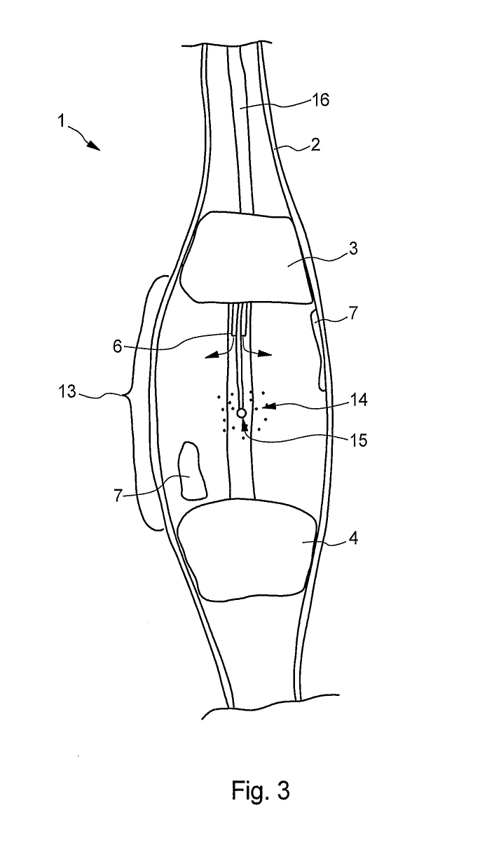

FIG. 3 shows a further device with a further arrangement of the aerosol generating apparatus 5 with the nozzle 15 and the insufflation apparatus 6.

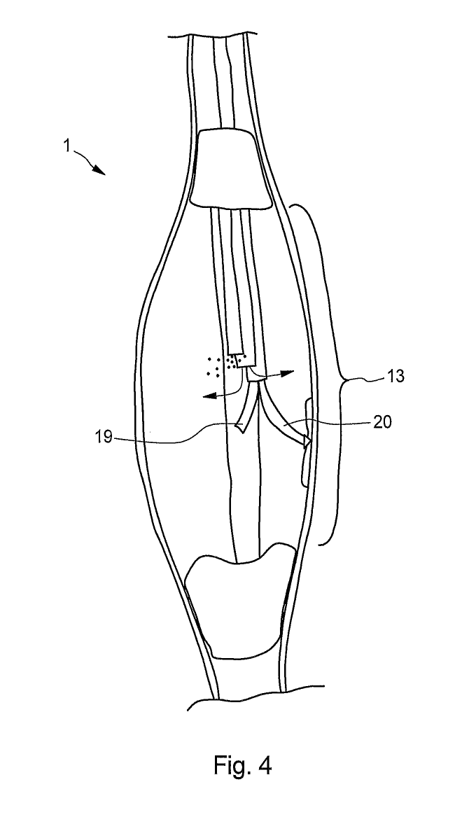

FIG. 4 shows the device 1 equipped with a monitoring means 19, in particular a camera positioned between the first and the second fluid chamber, suitable for monitoring the treatment section of the body cavity as well as an operation apparatus 20 suitable for manipulation of the wall of the treatment section of the body cavity.

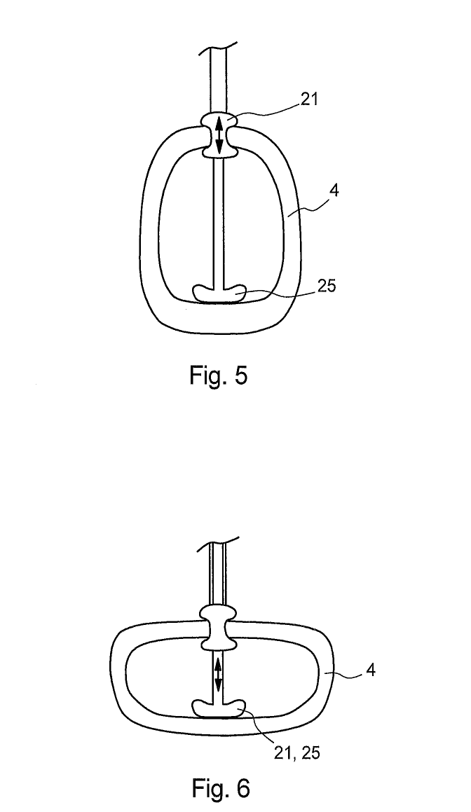

FIGS. 5 and 6 both show schematically how the length of at least one fluid chamber 4 may be adjusted using an adjustment means 21, as shown in FIG. 6, such as a telescopic device including a stamp 25 that has an impact on the fluid chamber.

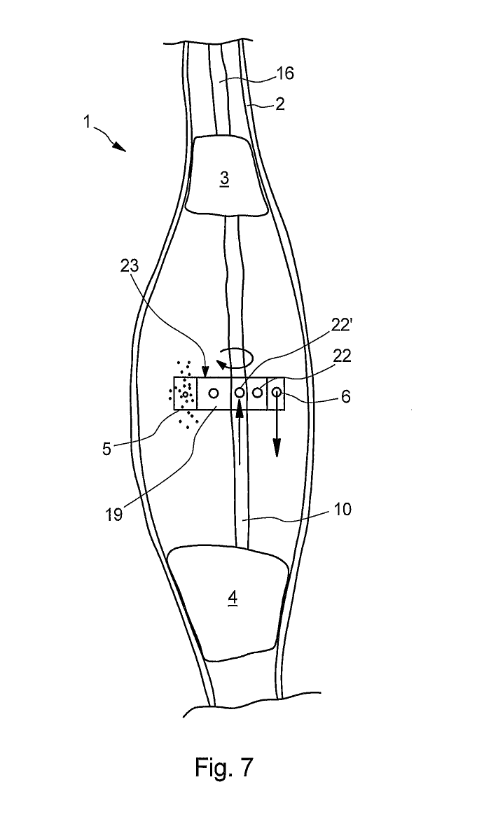

In FIG. 7, a further arrangement of an alternative device is schematically illustrated. In particular, the aerosol generating apparatus 5, the camera 19, a sensor 22, the insufflation apparatus 6 and a suction apparatus 22′ are shown positioned within a rotating element 23, which may be rotated independently from the position of the fluid chambers 3, 4.

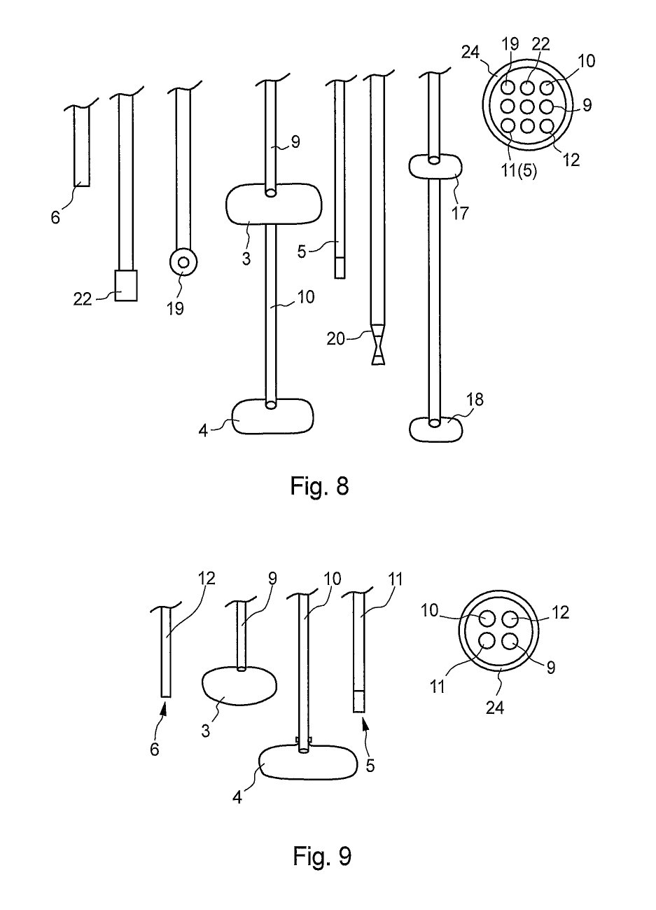

In FIG. 8, parts of an aerosol generating apparatus 5, the insufflation apparatus 6, the monitoring means 19, the sensor means 22, four fluid chambers 3, 4, 17, and 18, and an operation apparatus 20 with their respective supply lines are shown schematically, respectively. In particular, the common arrangement of the supply lines to the mentioned elements within an accommodation tube 24 that is positioned in front of the fluid chamber 3 is illustrated in a sectional view.

FIG. 9 shows parts of the device of FIG. 1, in particular the aerosol generating apparatus, the insufflation apparatus 6, and two fluid chambers 3, 4.

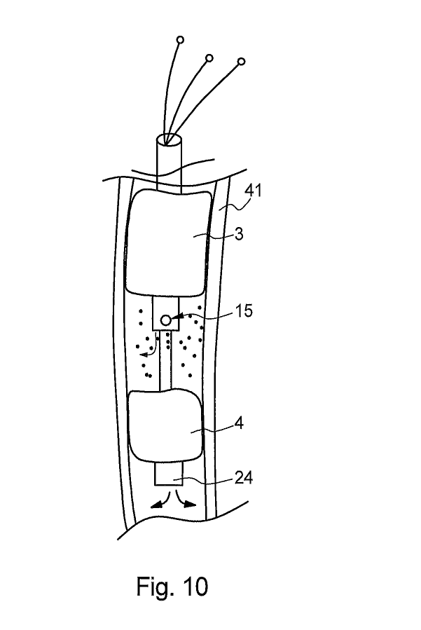

FIG. 10 shows a device for use in a further body cavity, namely the trachea 41. Illustrated is the implementation of an intubation means, such as hose 24 for the intubation of a patient through both fluid chambers 3, 4.

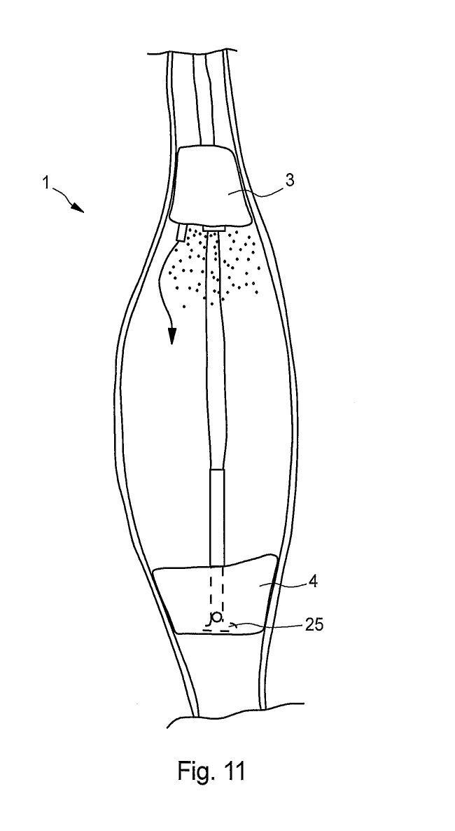

In FIG. 11 a device 1 is illustrated including an adjustable fluid chamber 4, which is adjustable with an adjustment means in the form of a stamp 25, as shown in FIG. 6.

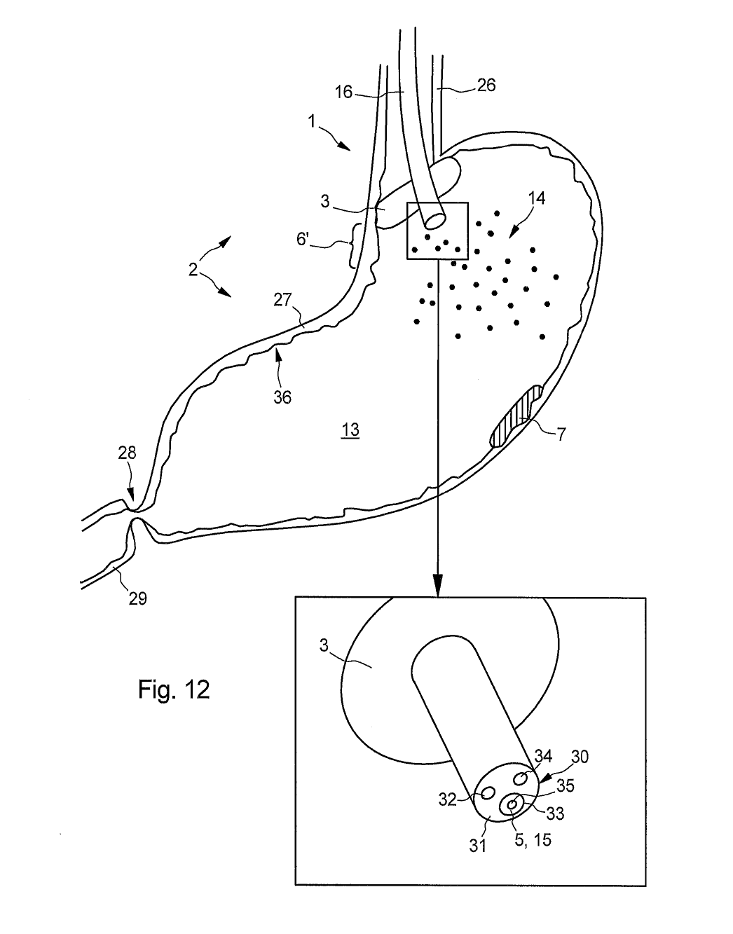

FIG. 12 shows a device 1 arranged within a body cavity 2 in form of the esophagus 26 and the stomach 27. Also shown is a pathological area in form of a tumor 7 within the stomach 27. The device 1 includes a fluid chamber 3 and a hose 16 that is passed through the fluid chamber. The fluid chamber 3 in form of a balloon seals the stomach 27 in the direction of the esophagus 26 forming a body cavity section 6′. The treatment section 13 is formed by the insufflated volume of the stomach 27 and is locally restricted from the pylorus 28 to the intestine 29. The hose 16 terminates in the treatment section 13 with an outlet means 30 having a planar formed front face 31. The outlet means 30 is illustrated in an enlarged view. The outer diameter of the outlet means is about 10 mm. Other diameters according to the size of the organs are possible. Typically, is a diameter of the outlet means that is used within the stomach not larger than 13 mm. At the front face 31 of the outlet means three openings 32 to 34 are formed. An opening 32 is provided for the supply of a gaseous medium, in this case CO₂, and is part of the insufflation apparatus 6. A second opening 33 is provided for the aerosol generating apparatus 5 in form of a nozzle 15 having an inner diameter of 20 micrometers. The third opening 34 is optional and is the end of a passage for passing through, for example, an operation tool, a sensor 22 or a fluid supply line or fluid drainage line. Not shown in detail is the connection of two fluid lines with the outlet means. In particular, the outlet means 30 forms a round trough-opening that terminates in a connection piece at the and face to which a insufflation hose can be connected. For connection with the nozzle 15, the outlet means 30 includes at the front face an internally threaded recess (not shown) that matches the outer thread of the nozzle for a threaded connection. Opposite from the front face 31, a further threaded connection is provided for the connection of the nozzle 15 with a pressure hose. During the treatment of the treatment section 14 is the pressure hose under a relatively high pressure, for example 20 bar, filled with at least one chemotherapeutic agent in liquid form. The liquid is upon exit from the small nozzle opening 35 abruptly relieved of pressure and consequently within the previously through the insufflation apparatus with CO₂ expanded stomach atomized into a mist, the aerosol 14. I has surprisingly been found that the aerosol 14 can be spread very evenly inside the gastric volume and that the fluid in form of the chemotherapeutic agent can according evenly affect the inner surface 36 of the through insufflation expanded stomach. Optional can the aerosol 14 also be charged electrically positive, for example through application of corresponding potential electrodes, and thus exhibit a negative potential relative to the body of the patient. What is reached is that the aerosol 14 is distributed more evenly on the stomach walls 36 by means of the potential difference of the formed gradient field.

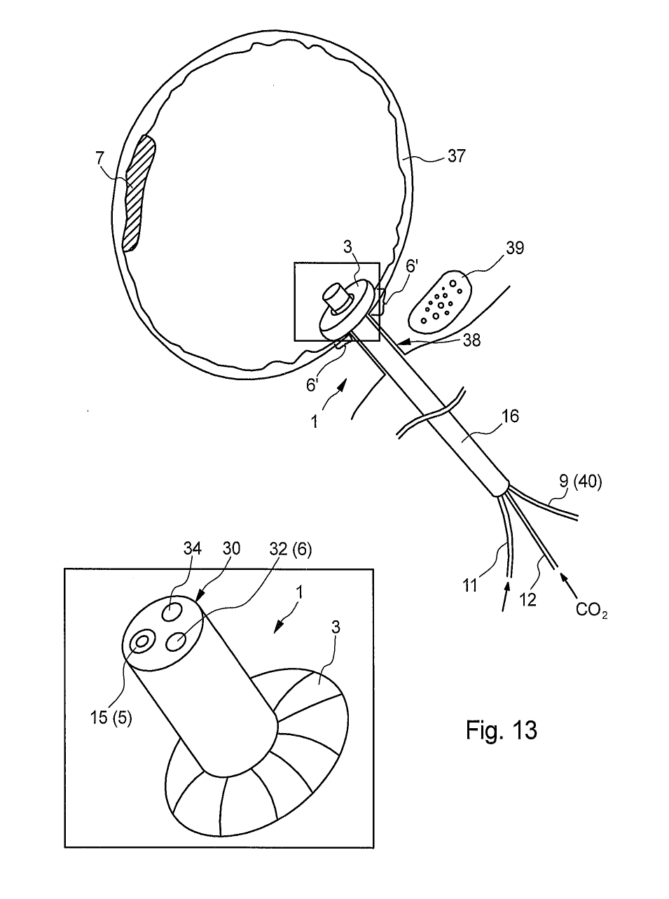

FIG. 13 shows a device 1 positioned within the body cavity 2 formed by the bladder 37 and the urethra 38 next to the pubis 39 in a schematic sectional representation. Shown is the closure of the bladder 37 within a section 6′ of the body cavity positioned within the bladder 37 opposite from the urethra 38 by means of an inflated fluid chamber 3 in form of a balloon filled with air. As in FIG. 12, an outlet means 30 is used that includes a nozzle 15 for the introduction of a chemotherapeutic agent via a high pressure hose 11, an optional opening 34 of an working channel and an opening 32 for the supply line 12 for supplying a fluid, such as CO₂, Helium or a gas mixture into the bladder 37 for its insufflation. Within the sheath tube 16, which is in a sealed connection with the outlet means 30, that is passed through the fluid chamber 3, the high pressure hose 11 and the CO₂-line 12 for insufflation are guided. An air hose 40 for filling the balloons can as shown be realized either as supply line 9 within the sheath tube 16 or outside of the sheath tube 16 directly connected with the fluid chamber 3. in the shown enlargement of a detail of FIG. 13, a further working channel terminates with an opening 34 at the front face 31 of the outlet means 30. This working channel can be eliminated or one or more additional working channels or passages may be positioned at the front face of the outlet means. The outlet means 30 may have an outer diameter of at most 10 mm. Smaller diameter of about 5 to 7 mm are typical when using the device within the bladder 38.

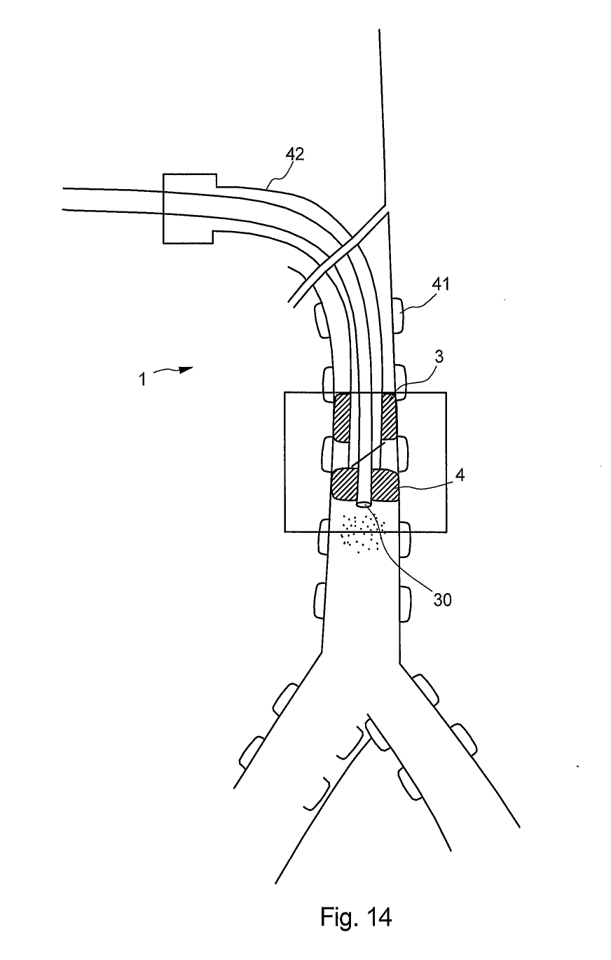

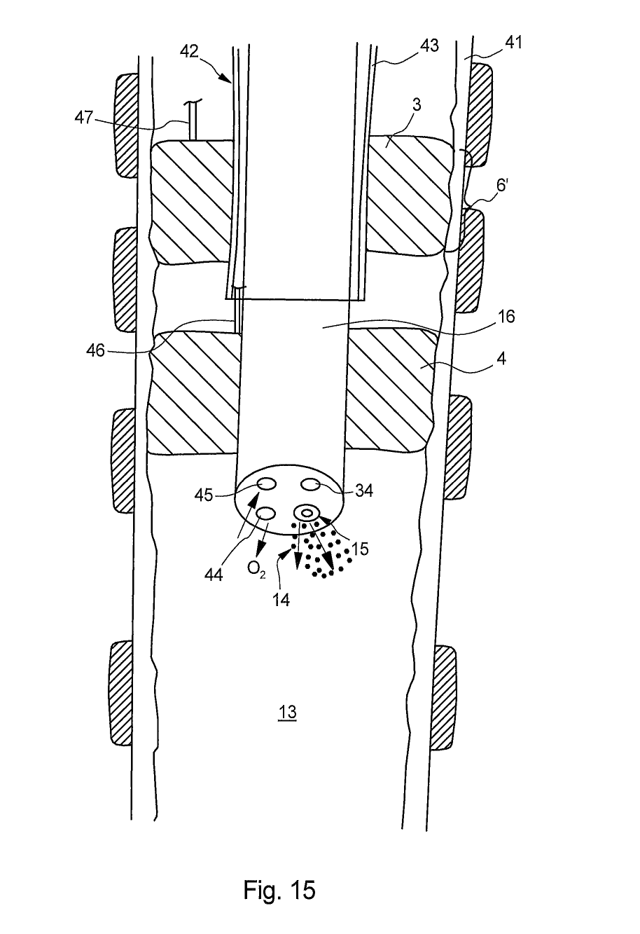

FIG. 14 and FIG. 15 show schematically a device 1 arranged within a body cavity in form of the trachea 41. The device includes here a endotracheal tube 42 with a first fluid chamber 3 in form of a small balloon and a hose 43 through which is typically ventilated. In addition to the ventilation via gas supply with oxygen or a gas mixture and gas removal, the administration of an aerosol via a nozzle is here in accordance with an embodiment of the invention also realized. Optionally, the air supply 46 for an additional fluid chamber 4 and also optionally the passing of optional means through the opening 34 of an working channel into the treatment section 13 can be realized. The air supply 47 of the first chamber 3 is typically realized outside of the shown hose 43. The air supply 46 to the further fluid chamber 4 can also be realized outside of the hose and outside of the sheath tube 16 that is guided through the hose.

It will be understood that the supply line to the fluid chamber 4 can be realized outside of the sheath tube 16 that is guided through the fluid chamber 3. It is further understood that in terms of the gas discharge 45, which can be enriched with the aerosol 14, the ventilator, not shown here, which is not connected with the gas discharge 45, adequate retention means should be provided, such as filter or condensation devices, to prevent the contamination of the areas surrounding the treatment section 13, here the trachea 41 and the lungs. Moreover, in a further embodiment, the second fluid chamber 4 may be omitted, it is ensured that no gas can pass the section 6′ sealed by the first fluid chamber 3.

1. A device suitable for insertion into and for use within a body cavity, especially an organ of a patient, the device comprising:
a first expandable fluid chamber for sealing a first section of the body cavity,
an aerosol generating apparatus for generation of a therapeutic aerosol within a treatment section of the body cavity positioned adjacent to the first section, and
an insufflation apparatus for insufflation of a gaseous medium into the treatment section of the body cavity.

2. The device according to claim 1, further including a second expandable fluid chamber for sealing a second section of the body cavity, wherein the treatment section is provided as a central section between the first and the second section.

3. The device according to claim 1, further including at least one item selected from the group of:
monitoring means suitable for monitoring the treatment section of the body cavity, wherein the monitoring means includes a camera,
sensor means suitable for detecting the aerosol present in the treatment section, and
operation apparatus suitable for manipulation of the wall of the treatment section of the body cavity.

4. The device according to claim 1, wherein a supply line of the aerosol generating apparatus is guided through the first fluid chamber into the treatment section of the body cavity.

5. The device according to claim 1, wherein at least one additional supply line is guided through the first fluid chamber into the treatment section of the body cavity.

6. The device according to claim 1, further including an intubation apparatus, wherein a supply line of the intubation apparatus is guided through the first fluid chamber.

7. The device according to claim 6, wherein a pressure between about 5 mbar and about 30 mbar, and preferably between about 10 mbar and about 15 mbar, is generated with the intubation apparatus.

8. The device according to claim 1, wherein the length of at least one of the fluid chambers is adjustable using adjustment means.

9. The device according to claim 1, wherein the distance between two fluid chambers is changeable using a telescopic device.

10. A device for drug therapy of a patient, the device comprising at least one fluid chamber and the device being suitable for executing a method comprising the steps of:
inserting the device into a body cavity of a patient,
introducing a fluid to a first fluid chamber, and
introducing an aerosol to a treatment section of the body cavity positioned adjacent to the first fluid chamber.

11. The device according to claim 1, further including an outlet means including at least an opening cross-section of the insufflation apparatus and a nozzle of the aerosol generating apparatus, wherein the outlet means opens into the treatment section.

12. The device according to claim 1, wherein the outlet means further includes at least one further passage leading into the treatment section.

13. The device according to claim 1, wherein the first fluid chamber is feedable through a trocar.

14. A method for using a device for drug therapy of a patient, the device comprising at least one fluid chamber, said method comprising the steps of:
inserting the device into a body cavity of a patient,
introducing a fluid to a first fluid chamber, and
introducing an aerosol to a treatment section of the body cavity positioned adjacent to the first fluid chamber.

15. The method according to claim 14, wherein the device further includes a second expandable fluid chamber for sealing a second section of the body cavity, wherein the treatment section is provided as a central section between the first and the second section.
